(19) United States
(12) Patent Application Publication
(10) Pub. No.: US 2017/0296127 A1
(43) Pub. Date: Oct. 19, 2017

(54) METHOD AND APPARATUS FOR GIVING A MEASUREMENT OF QUALITY FOR IMPEDANCE BASED RESPIRATION MONITORING

(71) Applicant: GENERAL ELECTRIC COMPANY, Schenectady, NY (US)

(72) Inventor: Panu Antero Takala, Helsinki (FI)

(21) Appl. No.: 15/485,963

(22) Filed: Apr. 12, 2017

(30) Foreign Application Priority Data
Apr. 13, 2016 (GB) 1606365.3

(51) Int. Cl. A61B 5/00; A61B 5/053; A61B 5/08

(52) U.S. Cl. CPC A61B 5/7203; A61B 5/0809; A61B 5/053; A61B 5/0531

(57) ABSTRACT

A method for giving a measurement of quality for impedance based respiration monitoring is disclosed. The method comprises attaching a lead to a subject, the lead being connected to a monitor, and receiving a signal from the lead, the lead being used for impedance respiration monitoring; estimating amplitude of cardiovascular artifact, CVA, derived from the signal of the lead; estimating amplitude of respiration derived from the signal of the lead; calculating a quality factor for the lead based on the estimated amplitude of CVA and the estimated amplitude of respiration; and indicating the quality factor for the lead to the user via the monitor.

### TECHNICAL FIELD

The present disclosure relates to respiration monitoring. More particularly, the present disclosure relates to a method and an apparatus for giving a measurement of quality for impedance based respiration monitoring. More particularly, the present disclosure relates to selection of an optimal lead for impedance based respiration monitoring.

### BACKGROUND

Electrical impedance is the measure of the opposition that a circuit presents to a current when a voltage is applied. In quantitative terms, it is the complex ratio of the voltage to the current in an alternating current (AC) circuit. Impedance extends the concept of resistance to AC circuits, and possesses both magnitude and phase, unlike resistance, which has only magnitude. When a circuit is driven with direct current (DC), there is no distinction between impedance and resistance; the latter can be thought of as impedance with zero phase angle.

When monitoring respiration using impedance based monitoring a lead is used. A lead is a group of wires and electrodes arranged in a specific way to give a picture, a view, of impedance of the thorax from a particular angle across the body, obtained by using different combinations of the electrodes attached to the body. There are standards for leads, for example, lead I, lead II, lead III, lead aVR, and lead aVL, etc. Often it has to be decided by the user which lead to use to get the best monitoring. Trial and error is often used to find which lead would be the optimal lead in different respiration monitoring situations.

### BRIEF DESCRIPTION

The present disclosure is directed to a method and an apparatus for giving a measurement of quality for impedance based respiration monitoring. This can be achieved by the features as defined by the independent claims. Further enhancements are characterized in the dependent claims.

According to one embodiment, the present disclosure is directed to a method for giving a measurement of quality for impedance based respiration monitoring. The method comprises: attaching a lead to a subject, the lead being connected to a monitor, and receiving a signal from the lead, the lead being used for impedance respiration monitoring; estimating amplitude of cardiovascular artifact, CVA, derived from the signal of the lead; estimating amplitude of respiration derived from the signal of the lead; calculating a quality factor for the lead based on the estimated amplitude of CVA and the estimated amplitude of respiration; and indicating the quality factor for the lead to the user via the monitor.

According to one embodiment, the present disclosure is directed to an apparatus for giving a measurement of quality for impedance based respiration monitoring. The apparatus comprises a monitor connectable to a subject via a lead. The monitor being configured to indicate a quality factor for the lead by: estimating amplitude of cardiovascular artifact, CVA, derived from a signal of the lead; estimating amplitude of respiration derived from the signal of the lead; calculating the quality factor for the respective lead based on the estimated amplitude of CVA and the estimated amplitude of respiration; and indicating the quality factor for the lead to the user via the monitor.

According to one embodiment, the quality factor is calculated as the estimated amplitude of respiration divided by the estimated amplitude of CVA, or vice versa. According to one embodiment, the quality factors for different leads are calculated in sequence, and an optimal lead for the impedance based respiration monitoring is indicated to the user via the monitor based on the quality factors. According to one embodiment, the quality factors for different leads are calculated simultaneously, and an optimal lead for the impedance based respiration monitoring is indicated to the user via the monitor based on the quality factors.

According to one embodiment, the indication of the quality factor to the user comprises informing the user to reposition an electrode of the lead on the subject and/or to ensure that an electrode of the lead is correctly applied to the subject. According to one embodiment, the quality factor is calculated for each lead, and the monitor indicates the quality factor for each lead. According to one embodiment, the signal may be scaled up according to the quality factor and displaying the scaled up signal of the lead to the user via the monitor.

At least one embodiment disclosed herein provides a method for giving a measurement of quality for impedance based respiration monitoring. At least one embodiment disclosed herein provides and an apparatus for giving a measurement of quality for impedance based respiration monitoring. At least one embodiment disclosed herein provides a method how medical staff using a monitor can ensure that an optimal lead is used for respiration monitoring of a subject, and an apparatus for this. At least one embodiment disclosed herein provides a solution that is inexpensive and easy to realize in reality. At least one of the embodiments disclosed herein provides one or more solutions to the problems and disadvantages with the background art. At least one embodiment avoids compromising accuracy of impedance respiration measurement caused by using the wrong lead. At least one embodiment provides improved patient safety and/or improved quality of respiration monitoring.

Other technical advantages of the present disclosure will be readily apparent to one skilled in the art from the following description and claims. Various embodiments of the present application obtain only a subset of the advantages set forth. No one advantage is critical to the embodiments. Any disclosed embodiment may be technically combined with any other disclosed embodiment or embodiments.

### BRIEF DESCRIPTION OF THE DRAWINGS

The accompanying drawings illustrate presently exemplary embodiments of the disclosure and serve to explain, by way of example, the principles of the disclosure.

FIG. 1 is a diagrammatic illustration of an apparatus according to an exemplary embodiment of the disclosure;

FIG. 2 shows a flow chart of a method according to an exemplary embodiment of the disclosure;

FIG. 3 is a diagram illustrating good quality respiration signal;

FIG. 4 is a diagram illustrating low quality respiration signal; and

FIG. 5 is a diagram illustrating poor quality respiration signal.

### DETAILED DESCRIPTION

In an impedance pneumographic signal, an impedance respiration, ImpResp, signal, both breaths and heart beats, cardiovascular artifact, CVA, of a subject are visible. Using a signal processing algorithm on the ImpResp measurement seeks to discard the heart beats to provide a reliable measurement of the patient's respiration rate, and potential cessation of breathing, apnea. The present disclosure uses amplitude of the cardiovascular artifact to provide a quantitative quality measure of the respiration signal in the lead that is used for ImpResp monitoring. The quality measure can be used for instructing the clinical staff to select the optimal lead for ImpResp measurement, or the selection can be done automatically, for example with measurement hardware configured to output signals from several leads simultaneously.

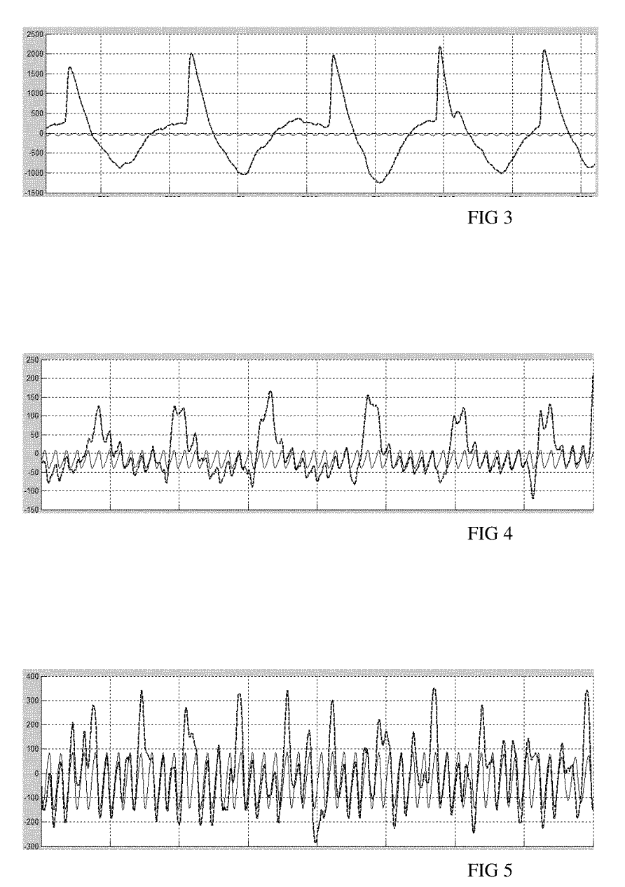

An indication of quality can be established based on separating the signal components of cardiac activity and breathing activity in the ImpResp signal, and comparison of their amplitudes. Signal processing methods estimate amplitude and shape of the cardiovascular artifact (CVA) in the ImpResp signal. Once estimated, the artifact can be subtracted from the raw signal to obtain the respiration signal. FIGS. 3 to 5 illustrate three ImpResp signals, and estimated CVA signals. The bold dotted line illustrates respiration and the other line illustrates CVA. In FIG. 3, respiration has high amplitude when compared to the CVA. This illustrates a good quality respiration signal, because the two signals can easily be separated. In FIG. 4, the CVA has almost the same amplitude as the respiration. This illustrates a lower quality respiration signal, because the two signals can not be as easily separated from each other as in FIG. 3. In FIG. 5, detection of breaths from the original signal is more prone to errors when compared to the signal in FIG. 3. An estimation of the CVA, for example, is not perfect and remains of the true artifact may be left in the signal after subtraction of an estimate from the raw signal. Also, artifacts caused by ectopic heart beats have a different shape and amplitude than normal beats and can not be completely removed. Therefore, the higher the amplitude separation between breaths and heart beats in the raw signal, the better performance of the measurement.

The present disclosure provides a measure of quality for the ImpResp signal. The measure of quality, Q, may be the ratio of breath and cardiovascular artifact amplitudes, for example: Q=amplitude (breath)/amplitude (CVA). Amplitude of breath may by its own be a good indicator of ImpResp signal quality. However, as can be noted by comparing the signals from FIGS. 4 and 5, the signal with lower respiration amplitude, as in FIG. 4, can have better quality than a signal with higher respiration amplitude if amplitude of CVA is very high, as in FIG. 5.

The broadest scope of this disclosure is that the measure of quality is based only on the amplitude of breath. All embodiments disclosed herein may use only the amplitude of breath for establishing a measurement of quality. For example, a method for giving a measurement of quality for impedance based respiration monitoring may comprise: attaching a lead to a subject, the lead being connected to a monitor, and receiving a signal from the lead, the lead being used for impedance respiration monitoring; estimating amplitude of respiration derived from the signal of the lead; calculating a quality factor for the lead based on the estimated amplitude of respiration; and indicating the quality factor for the lead to the user via the monitor. For example, an apparatus for giving a measurement of quality for impedance based respiration monitoring may comprise a monitor connectable to a subject via a lead, the monitor being configured to indicate a quality factor for the lead by: estimating amplitude of respiration derived from the signal of the lead; calculating the quality factor for the respective lead based on the estimated amplitude of respiration; and indicating the quality factor for the lead to the user via the monitor. All other embodiments disclosed herein may be made correspondingly dependent on such a method or apparatus. However, the claimed embodiments are limited to estimating quality not only using the amplitude of one of ImpResp and CVA, but comparing both the amplitudes.

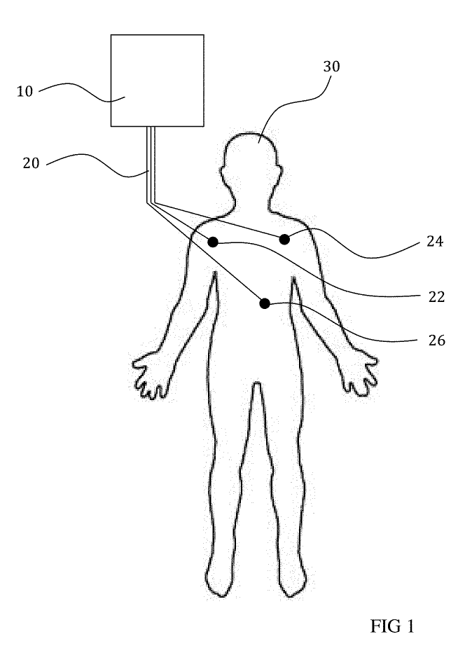

Turning to FIG. 1, according to an embodiment, an impedance respiration monitor 10 measures the impedance between electrodes 22, 24, 26 that are attached to a subject 30. The monitor measures between the electrodes, for example between a drive electrode 22 and a receive electrode 24, to monitor air flow in lungs. As the subject 30 inhales, air, which is an insulator, enters the lungs and causes the net impedance in the circuit to increase. When the subject exhales, air leaves the lungs and causes the impedance in the circuit to decrease. Thus, a high-frequency AC current is injected into the tissue through the drive electrode 22. The AC current causes a potential difference to develop across the drive electrode 22 and the receive electrode (voltage sensing electrode) 24. A lead 20 is a group of wires and electrodes 22, 24, 26 arranged in a specific way to give a picture, a view, of the electrical activity (impedance) of the chest and the heart from a particular angle across the body of the subject 30, obtained by using different combinations of these wires. Examples of leads are lead I, lead II, lead III, lead aVR, and lead aVL. For example, lead I is from the right to the left arm, and lead II from the right arm to the left leg. A lead comprises at least two points, electrodes, and measures impedance between the two points. The lead 20 may be connected by wires to the monitor 10 as in FIG. 1, but may instead, or in addition, be connected wirelessly to the monitor 10. It is a problem to find the lead that provides optimal monitoring.

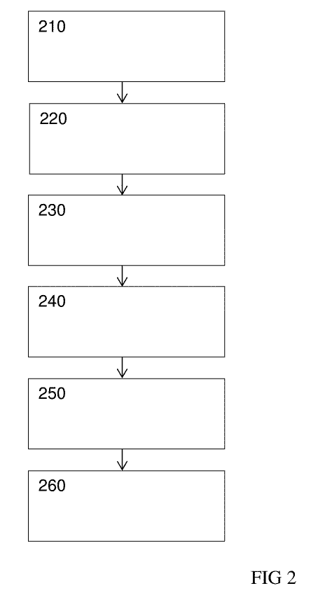

Turning to FIG. 2, a method for giving a measurement of quality for impedance based respiration monitoring is disclosed. The method comprises attaching a lead to a subject, the lead being connected to a monitor, and receiving a signal from the lead, the lead being used for impedance respiration monitoring. This step is illustrated in FIG. 2 by reference 210. The next two steps that can be made in any order is estimating amplitude of cardiovascular artifact, CVA, derived from the signal of the lead, and estimating amplitude of respiration derived from the signal of the lead. These two steps are illustrated in FIG. 2 by reference 220 and 230, respectively. The next step is calculating a quality factor for the lead based on the estimated amplitude of CVA and the estimated amplitude of respiration. This step is illustrated in FIG. 2 by reference 240. The next step is indicating the quality factor for the lead to the user via the monitor. This step is illustrated in FIG. 2 by reference 250. As explained earlier, the estimates are done by monitor software, firmware, and/or hardware for getting the amplitudes from the signal. While the measure of quality is established by using both the amplitudes, as described above, a corresponding method for giving a measurement of quality may use only the amplitude of respiration. The method provides a means for selecting an optimal lead for ImpResp measurement. Establishing the optimal lead ensures and improves quality of respiration rate and apnea monitoring, less false alarms are given, and apnea episodes are detected with better accuracy. Also, improved quality of respiration rate measurement increases customer satisfaction and gives monitors a higher value.

According to one embodiment, the quality factor is calculated as the estimated amplitude of respiration divided by the estimated amplitude of CVA. According to another embodiment, the quality factor is calculated as the estimated amplitude of CVA divided by the estimated amplitude of respiration. The quality factor is thus a ratio of the two. An optimal lead is the lead giving the best quality factor, for example lead II or lead III. If the quality factor is calculated as the estimated amplitude of respiration divided by the estimated amplitude of CVA, then the optimal lead is the lead with the highest quality factor. If the quality factor is calculated as the estimated amplitude of CVA divided by the estimated amplitude of respiration, then the optimal lead is the lead with the lowest quality factor.

According to one embodiment, the quality factors for different leads are sequentially calculated, and an optimal lead for the impedance based respiration monitoring is indicated to the user via the monitor based on the quality factors. The quality factor for each possible leads may be calculated one after the other, depending on the electrodes of the lead. By sequential measuring of each lead connected to the monitor and subject a quality factor may be established for each possible lead. Based on the quality factors a user may select an optimal lead. The quality factors may not have to be indicated to the user and the monitor may instead indicate the optimal lead by selecting the optimal lead for the user. As explained above, the optimal lead is the lead giving the best quality factor.

According to one embodiment, the quality factors for different leads are simultaneously calculated, and an optimal lead for the impedance based respiration monitoring is indicated to the user via the monitor based on the quality factors. The quality factor for each possible leads may be calculated all at the same time, depending on the electrodes of the lead. By simultaneously measuring all leads connected to the monitor and subject a quality factor may be established for each possible lead. Based on the quality factors a user may select an optimal lead. The quality factors may not have to be indicated to the user and the monitor may instead indicate the optimal lead by selecting the optimal lead for the user. As explained above, the optimal lead is the lead giving the best quality factor.

According to one embodiment, the method may further comprise monitoring respiration of the subject using the optimal sensor. In this way respiration monitoring may be made of the subject and the best possible lead may be used for such monitoring.

According to one embodiment, the indication of the quality factor to the user comprises informing the user to reposition an electrode of the lead on the subject. A poor quality of the respiration monitoring may result from a poorly, or incorrectly, placed electrode of the lead to the subject. The indication may include a message that the user of the monitor should check and/or reposition a specific electrode of a lead to get a better quality factor.

According to one embodiment, the indication of the quality factor to the user comprises informing the user to ensure that an electrode of the lead is correctly applied to the subject. A poor quality of the respiration monitoring may result from a poorly, or incorrectly, attached electrode of the lead to the subject. The indication may include a message that the user of the monitor should check and/or correctly apply a specific electrode of a lead to get a better quality factor.

According to one embodiment, the quality factor is calculated for each lead, and the monitor indicates the quality factor for each lead. By the monitor indicating all the calculated quality factors for the different possible leads, a user may view them and may decide what lead or combination of leads to use.

According to one embodiment, the method may further comprise scaling up the signal according to the quality factor and displaying the scaled up signal of the lead to the user via the monitor. This step is illustrated in FIG. 2 by reference 260. Hereby, the quality factor may decide the default scaling on the monitor. Many monitors simply show any signal as large as they can on a screen. However, that results in that a user can not easily see the quality of the signal. Embodiments in this disclosure ensure that the quality is also indicated on the monitor, not only the signal as large as possible. In addition, a corresponding scaling may then be made based on the quality factor.

According to one embodiment, an apparatus for giving a measurement of quality for impedance based respiration monitoring comprises a monitor connectable to a subject via a lead. The monitor is configured to indicate a quality factor for the lead by: estimating amplitude of cardiovascular artifact (CVA) derived from a signal of the lead; estimating amplitude of respiration derived from the signal of the lead; calculating the quality factor for the respective lead based on the estimated amplitude of CVA and the estimated amplitude of respiration; and indicating the quality factor for the lead to the user via the monitor. While the measure of quality is established by using both the amplitudes, as described above, a corresponding apparatus for giving a measurement of quality may use only the amplitude of respiration. The apparatus provides a means for selecting an optimal lead for ImpResp measurement. Establishing the optimal lead ensures and improves quality of respiration rate and apnea monitoring, less false alarms are given, and apnea episodes are detected with better accuracy. Also, improved quality of respiration rate measurement increases customer satisfaction and gives monitors a higher value.

According to one embodiment, the quality factor is calculated as the estimated amplitude of respiration divided by the estimated amplitude of CVA. According to another embodiment, the quality factor is calculated as the estimated amplitude of CVA divided by the estimated amplitude of respiration. The quality factor is thus a ratio of the two. An optimal lead is the lead giving the best quality factor, for example lead II or lead III. If the quality factor is calculated as the estimated amplitude of respiration divided by the estimated amplitude of CVA, then the optimal lead is the lead with the highest quality factor. If the quality factor is calculated as the estimated amplitude of CVA divided by the estimated amplitude of respiration, then the optimal lead is the lead with the lowest quality factor.

According to one embodiment, the quality factors for different leads are sequentially calculated, and an optimal lead for the impedance based respiration monitoring is indicated to the user via the monitor based on the quality factors. The monitor may be configured to calculate and indicate the quality factors as disclosed above to the corresponding method.

According to one embodiment, the quality factors for different leads are simultaneously calculated, and an optimal lead for the impedance based respiration monitoring is indicated to the user via the monitor based on the quality factors. The monitor may be configured to calculate and indicate the quality factors as disclosed above to the corresponding method.

Calculating and indicating the quality factor according to these two last mentioned embodiments, the quality factor for each possible leads may be calculated depending on the electrodes of the lead. By sequential or simultaneously measuring of each lead connected to the monitor and subject a quality factor may be established for each possible lead. Based on the quality factors a user may select an optimal lead. The quality factors may not have to be indicated to the user and the monitor may instead indicate the optimal lead by selecting the optimal lead for the user. As explained above, the optimal lead is the lead giving the best quality factor.

According to one embodiment, the monitor is further configured to monitor respiration of the subject using the optimal lead. In this way respiration monitoring may be made of the subject and the best possible lead may be used for such monitoring.

According to one embodiment, the monitor is further configured to indicate the quality factor to the user with information to the user to reposition an electrode of the lead on the subject. The monitor may be configured to indicate that the user should select another position for one or more of the electrodes of the lead to improve the quality.

According to one embodiment, the monitor is further configured to indicate the quality factor to the user with information to the user to ensure that an electrode of the lead is correctly applied to the subject. The monitor may be configured to indicate that the user should check that the one or more of the electrodes of the lead are attached properly to the subject to improve the quality.

According to one embodiment, the monitor is further configured to calculate the quality factor for each lead, and the monitor is configured to indicate the quality factor for each lead. Thus a calculation of the quality factors for each possible lead allows the monitor to indicate the quality factor for each possible lead, or a selected range of leads. This corresponds to the corresponding embodiment of the method mentioned above. By the monitor indicating all the calculated quality factors for the different possible leads, a user may view them and may decide what lead or combination of leads to use.

According to one embodiment, the monitor is further configured to scale up the signal according to the quality factor and displaying the scaled up signal of the lead to the user via the monitor. This corresponds to step 260 illustrated in FIG. 2 and mentioned above. Hereby, the quality factor may decide the default scaling on the monitor. Many monitors simply show any signal as large as they can on a screen. However, that results in that a user can not easily see the quality of the signal. Embodiments in this disclosure ensure that the quality is also indicated on the monitor, not only the signal as large as possible. In addition, a corresponding scaling may then be made based on the quality factor.

In at least one embodiment, the method and apparatus gives a measurement of quality for respiration monitoring. At least on embodiments provides for selecting an optimal lead for ImpResp measurement, thereby improving quality of respiration rate and apnea monitoring, causing less false alarms, and apnea episodes are detected with better accuracy. Improved quality of respiration rate measurement increases customer satisfaction and promotes monitor sales. The disclosed advantages and technical advantages above apply both for the embodiments of the methods as well as the embodiments of the apparatus.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for giving a measurement of quality for impedance based respiration monitoring, the method comprising:
attaching a lead to a subject, the lead being connected to a monitor, and receiving a signal from the lead, the lead being used for impedance respiration monitoring;
estimating amplitude of cardiovascular artifact (CVA), derived from the signal of the lead;
estimating amplitude of respiration derived from the signal of the lead;
calculating a quality factor for the lead based on the estimated amplitude of CVA and the estimated amplitude of respiration; and
indicating the quality factor for the lead to the user via the monitor.

2. The method according to claim 1, wherein the quality factor is calculated as the estimated amplitude of respiration divided by the estimated amplitude of CVA, or vice versa.

3. The method according to claim 1, wherein the quality factors for different leads are calculated in sequence, and an optimal lead for the impedance based respiration monitoring is indicated to the user via the monitor based on the quality factors.

4. The method according to claim 1, wherein the quality factors for different leads are calculated simultaneously, and an optimal lead for the impedance based respiration monitoring is indicated to the user via the monitor based on the quality factors.

5. The method according to claim 1, wherein the indication of the quality factor to the user comprises informing the user to reposition an electrode of the lead on the subject.

6. The method according claim 1, wherein the indication of the quality factor to the user comprises informing the user to ensure that an electrode of the lead is correctly applied to the subject.

7. The method according claim 1, wherein the quality factor is calculated for each lead, and the monitor indicates the quality factor for each lead.

8. The method according to claim 1, further comprising scaling up the signal according to the quality factor and displaying the scaled up signal of the lead to the user via the monitor.

9. An apparatus for giving a measurement of quality for impedance based respiration monitoring, the apparatus comprising a monitor connectable to a subject via a lead, the monitor being configured to indicate a quality factor for the lead by:
estimating amplitude of cardiovascular artifact (CVA) derived from a signal of the lead;
estimating amplitude of respiration derived from the signal of the lead;
calculating the quality factor for the respective lead based on the estimated amplitude of CVA and the estimated amplitude of respiration; and
indicating the quality factor for the lead to the user via the monitor.

10. The apparatus according to claim 9, wherein the quality factor is calculated as the estimated amplitude of respiration divided by the estimated amplitude of CVA, or vice versa.

11. The apparatus according to claim 9, wherein the quality factors for different leads are calculated in sequence, and an optimal lead for the impedance based respiration monitoring is indicated to the user via the monitor based on the quality factors.

12. The apparatus according to claim 9, wherein the quality factors for different leads are calculated simultaneously, and an optimal lead for the impedance based respiration monitoring is indicated to the user via the monitor based on the quality factors.

13. The apparatus according claim 9, wherein the monitor is further configured to indicate the quality factor to the user with information to the user to reposition an electrode of the lead on the subject.

14. The apparatus according to claim 9, wherein the monitor is further configured to indicate the quality factor to the user with information to the user to ensure that an electrode of the lead is correctly applied to the subject.

15. The apparatus according to claim 9, wherein the monitor is further configured to calculate the quality factor for each lead, and the monitor is configured to indicate the quality factor for each lead.

16. The apparatus according to claim 9, wherein the monitor is further configured to scale up the signal according to the quality factor and displaying the scaled up signal of the lead to the user via the monitor.
